(19) United States
(12) Patent Application Publication
(10) Pub. No.: US 2017/0231747 A1
(43) Pub. Date: Aug. 17, 2017

(54) Extraluminal Urethral Prostate Shield Apparatus

(71) Applicant: Peter Sayet, Fort Lauderdale, FL (US)

(72) Inventor: Peter Sayet, Fort Lauderdale, FL (US)

(21) Appl. No.: 15/435,108

(22) Filed: Feb. 16, 2017

Related U.S. Application Data
(60) Provisional application No. 62/295,712, filed on Feb. 16, 2016.

(51) Int. Cl. A61F 2/04; A61F 2/82

(52) U.S. Cl. CPC A61F 2/04; A61F 2/82; A61F 2002/047; A61F 2230/0069

(57) ABSTRACT

An extraluminal urethral prostate shield apparatus includes at least two collar members positionable around at least a portion of a bodily lumen. A plurality of longitudinal members securable between opposing faces of each of the at least two collar members in parallel array, surrounds the bodily lumen whereby obtrusion of enlarged tissue against the lumen is preventable and luminal patency is maintained.

### CROSS-REFERENCE TO RELATED APPLICATIONS

This nonprovisional application for utility patent claims the benefit of provisional application no. 62295712 filed on Feb. 16, 2016

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### SPECIFICATION

I have invented new and useful improvements in an extraluminal urethral prostate shield apparatus as described in this specification and that I claim the benefit of provisional application no. 62/295,712 filed on Feb. 16, 2016.

### BACKGROUND OF THE INVENTION

Various types of apparatuses are known in the prior art directed to maintaining patency of the urethra. Most require insertion into the urethra and expansion of either a balloon apparatus or deployment of a catheter or stent into the urethra to duct urine out of the bladder and enable urination. Nearly all such devices are intraluminal. What is needed is an extraluminal urethral prostate shield apparatus disposed to exteriorly encircle the prostatic urethra and shield the urethra from obtrusion effected by enlargement of the prostate, for example.

The present invention, therefore, is installable to gird the prostatic urethra with minimal surgery required, wherein a first collar member is disposed girdling the urethra most proximal the bladder neck on a distal side of the prostate, and a second collar member is disposed girdling the prostatic urethra most proximal the membranous urethra on a proximal side of the prostate, whereby a plurality of longitudinal members is disposed arrayed in parallel interconnecting said first and second collars surrounding the prostatic urethra, thereby preventative of obtrusion of the prostatic urethra by enlargement of the prostate.

### FIELD OF THE INVENTION

The present invention relates to an extraluminal urethral prostate shield apparatus devised to maintain patency of ducts and lumens in the body by installation extraluminally around said duct or lumen. In the present disclosure, attention is given to extraluminal situation around the prostatic urethra between the prostatic urethra and prostate, however additional ducts and lumens are contemplated as part of this disclosure.

Benign prostatic hyperplasia (BPH) is a well known condition affecting as many as 14 million men in the United States (2010). More than 90% of men over age 80 suffer from the condition. Enlarging of the prostate obtrudes against the prostatic urethra rendering difficulty in expelling urine from the bladder. Moreover, post-operative swelling of the prostate subsequent various treatments for BPH and other urological conditions may likewise lead to obtrusion of the urethra.

To maintain patency of the prostatic urethra, a catheter or stent is typically inserted into the bladder through the lumen of the urethra. The prostatic urethra is thus opened from the inside, and a channel is created for the free flow of urine therethrough. Such male catheterization is one of the most frequent procedures performed by health professionals in the modern day. The present invention, on the other hand, is devised to fit exteriorly around the prostatic urethra to prevent direct contact of the prostate against the urethra. Enlargement of the prostate is thus disabled from obtruding the prostatic urethra and patency is maintained.

### SUMMARY OF THE INVENTION

The present extraluminal urethral prostate shield apparatus includes a first collar member, a plurality of longitudinal members, and a second collar member. Each of the first and second collar members is substantially alike; each is devised to girdle the prostatic urethra and retain the plurality of longitudinal members endwise inserted into at least one opposing surface, whereby the prostatic urethra is encaged by said plurality of longitudinal members disposed in parallel adjacent the urethra between the urethra and the prostate, and enlargement of the prostate is prevented from obtruding the urethra thereby.

Each of the first and second collar members includes a body portion having a primary pivot disposed thereupon. The primary pivot enables articulation of each of a first joining end and a second joining end between each of a first position and a second position. The first joining end fastens to the second joining end when the body portion is moved to the second position and the body portion is then disposed encircling a central aperture disposed therethrough.

Each of the first and second collar members further includes at least one opposing surface disposed on the body portion. The at least one opposing surface includes a plurality of insertion points serried around the central aperture, into each of which plurality of insertion points each of the plurality of longitudinal members is endwise insertable.

Thus the first collar member is surgically mounted to gird the prostatic urethra on a distal side of the prostate, proximal the bladder neck. When moved to the second position the first collar member is thus fastened around the urethra, said urethra disposed through the central aperture formed when the body member is moved to the second position.

Each of the plurality of longitudinal members is thence insertable into a corresponding one of the plurality of insertion points along a line parallel with, and adjacent to, the prostatic urethra. Each of the plurality of longitudinal members may be inserted alongside the prostatic urethra between the prostatic urethra and the prostate proper with minimal surgery required.

Once fitted endwise into each of the plurality of insertion points disposed upon the at least one opposing surface of the first collar member, the second collar member is thence surgically installable girding the urethra proximal the membranous urethra on a proximal side of the prostrate. The second collar member is thence closed and moved such that each of the plurality of longitudinal members is fit endwise into each of the plurality of insertion points disposed upon the at least one opposing surface of said second collar member. A cage is thus erectable surrounding the prostatic urethra and securable in a position to prevent obtrusion of the urethra by contact imposition of an enlarged prostate. Patency of the prostatic urethra is thus maintained extraluminally.

Thus has been broadly outlined the more important features of the present extraluminal urethral prostate shield apparatus so that the detailed description thereof that follows may be better understood and in order that the present contribution to the art may be better appreciated.

For better understanding of the extraluminal urethral prostate shield apparatus, its operating advantages and specific objects attained by its uses, refer to the accompanying drawings and description.

### BRIEF DESCRIPTION OF THE DRAWINGS

### FIGURES

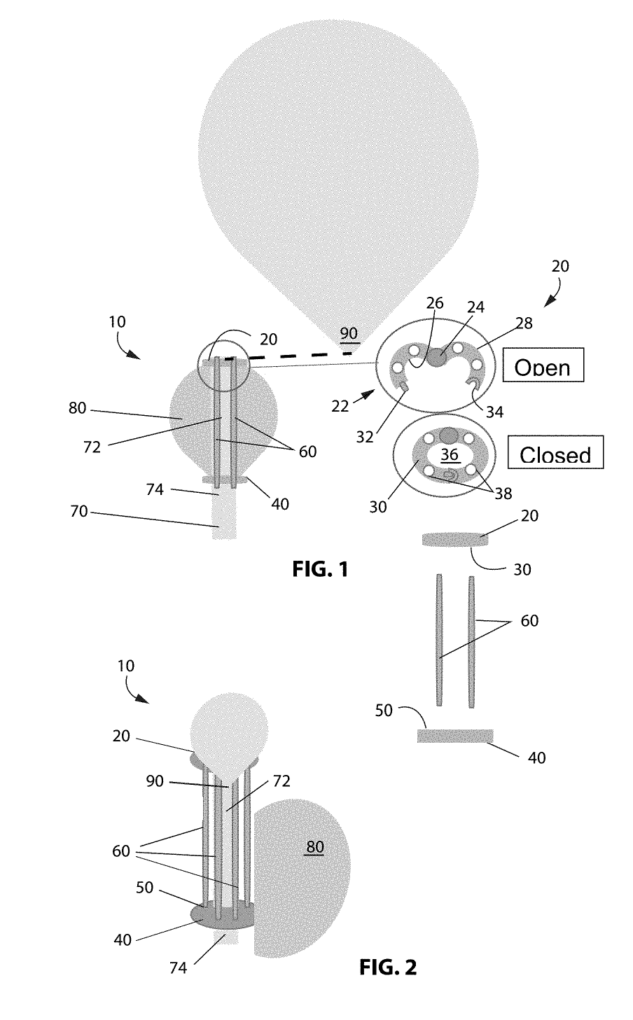

FIG. 1 is a plan view of an example embodiment.

FIG. 2 is a plan view of an example embodiment.

### DETAILED DESCRIPTION OF THE DRAWINGS

With reference now to the drawings, and in particular FIGS. 1 through 2 thereof, example of the instant extraluminal urethral prostate shield apparatus employing the principles and concepts of the present extraluminal urethral prostate shield apparatus and generally designated by the reference number 10 will be described.

Referring to FIGS. 1 through 2 a preferred embodiment of the extraluminal urethral prostate shield apparatus 10 is illustrated.

The present extraluminal urethral prostate shield apparatus 10 has been devised to maintain patency of a patient's urethra 70 by fit exteriorly mounted encircling the patient's urethra 70 and thus prevent inhibition of the urethra 70 by enlargement of the patient's prostate 80. A series of longitudinal members 60, mounted between each of a first and second collar member 20, 40, prevent direct contact of an enlarged prostate 80 with the prostatic urethra 72, and thereby maintain patency of the prostatic urethra 72 without intraluminal insertion.

The present extraluminal urethral prostate shield apparatus 10, therefore, includes a first collar member 20 fittable to girdle the prostatic urethra 72 in a position most proximal the bladder neck 90 on the distal side of the prostate 80. The first collar member 20 includes a primary pivot 24 disposed upon a body portion 22, an interior rim 26, an exterior rim 28, at least one opposing surface 30, a first joining end 32, and a second joining end 34 disposed endwise upon the body portion 22 to fasten with the first joining end 32 and bound a central aperture 36 formed when said first joining end 32 is fastened to said second joining end 34.

The first collar member 20 is therefore moveable between a first position, with each of said first and second joining ends 32, 34 disposed apart, and a second position, wherein the first joining end 32 fastens to the second joining end 34 and a central aperture 36 is encircled by the body portion 22 appropriate to accommodate the urethra 70 (or other duct) therethrough.

A plurality of insertion points 38 is disposed upon the at least one opposing surface 30 of the body portion 22, into each of which insertion points 38 each of a plurality of longitudinal members 60 is endwise insertable and thence maintainable therein in parallel relation projected arrayed perpendicularly extended from the at least one opposing surface 32. Each of the plurality of longitudinal members 60, therefore, is disposed lengthwise bounding the urethra 70 when inserted into, and retained by, the first collar member 20 disposed in the second position.

A second collar member 40 is thence fittable girdling the prostatic urethra 72 most proximal the membranous urethra 74 on a proximal side of the prostate 80. The second collar member 40 is substantially similar to the first collar member 20, and therefore likewise moveable to a second position to encircle the urethra 70 and thence accommodate and retain each of the plurality of longitudinal members 60 endwise into at least one opposing surface 50 disposed oriented opposite the at least one opposing surface 30 of the first collar member 20.

Each of the plurality of longitudinal members 60 is therefore positional oriented lengthwise between each of the first and second collar members 20, 40, disposed in parallel array surrounding the prostatic urethra 72 between the urethra 70 and prostate 80. The plurality of longitudinal members 60 thus comprises a shield preventing obtrusion by the prostate 80 whereby patency of the urethra 70 is enabled and maintained.

It should be noted that the present disclosure is contemplated for use with really any duct wherein patency is desirable and effective by extraluminal situation of the present extraluminal prostate shield apparatus 10. Although the present disclosure is directed primarily for use maintaining patency of the prostatic urethra in male patients, the device is nonetheless considerable for maintaining patency in any duct wherein swelling or obtrusion of surrounded tissue is preventable by direct inhibition of contact with the duct when shielded by the present device 10.

What is claimed is:

1. An extraluminal urethral prostate shield apparatus comprising;
at least a first collar member comprising;
a body portion;
an opposing surface disposed upon the body portion;
a plurality of insertion points disposed upon the opposing surface;
a central aperture disposed encircled within the body portion;
at least a second collar member comprising;
a body portion;
an opposing surface disposed upon the body portion, facing the first collar member;
a plurality of insertion points disposed upon the opposing surface;
a central aperture disposed encircled within the body portion; and
a plurality of longitudinal members parellely positionable between the opposing surfaces of each of the plurality of collar members, each of said plurality of longitudinal members insertable endwise into a corresponding one of the plurality of insertion points thereof;
wherein the plurality of collar members is interconnected by the plurality of longitudinal members, protectively encaging the central apertures to gird a lumen of a patient therein, whereby obtrusion of neighboring enlarged tissue is preventable, and luminal patency is maintained.

2. The extraluminal urethral prostate shield apparatus of claim 1 wherein each of the plurality of collar members further comprises;
a first joining end; and
a second joining end.

3. The extraluminal urethral prostate shield apparatus of claim 2 wherein the first joining end and the second joining end are connectable, whereby each of the first and second collar members is fastenable.

4. The extraluminal urethral prostate shield apparatus of claim 3, wherein each of the plurality of collar members further comprises;
a primary pivot disposed centrally upon the body portion between the first joining end and the second joining end.

5. The extraluminal urethral prostate shield apparatus of claim 4, wherein the body portion of each of the plurality of collar members is moveable between a open position, with the first and the second joining ends disposed apart, and a closed position, with the first joining end fastened to the second joining end, whereby the central aperture is enclosable by said body portion.

6. An extraluminal urethral prostate shield apparatus comprising;
a plurality of collar members, each of said collar members comprising;
a body portion comprising;
an interior rim disposed internally on the body portion;
an exterior rim disposed peripherally on the body portion;
a primary pivot disposed centrally upon the body portion;
each of a first joining end and a second joining end articulated by the primary pivot, each of said first and second joining ends endwise delimiting the body portion and
moveable between an open position, whereat each of said first and second joining ends is disposed apart, and a closed position, whereat each of the first joining end and the second joining end is disposed conjoined;
an opposing surface disposed upon the body portion;
a plurality of insertion points disposed serried upon the opposing surface of body portion;
a central aperture disposed bounded by the interior rim of the body portion; and
a plurality of longitudinal members parellely positionable between the opposing surfaces of each of the plurality of collar members, each of said plurality of longitudinal members insertable endwise into a corresponding one of the plurality of insertion points thereof;
wherein the plurality of collar members is interconnected by the longitudinal members, whereby a lumen of a patient is encaged, and obtrusion of neighboring enlarged tissue is preventable, and luminal patency is maintained.
